(19) United States
(12) Patent Application Publication
(10) Pub. No.: US 2019/0049400 A1
(43) Pub. Date: Feb. 14, 2019

(54) ELECTRODES, AND METHODS OF USE IN DETECTING EXPLOSIVES AND OTHER VOLATILE MATERIALS

(71) Applicant: Razzberry, Inc., New Haven, CT (US)

(72) Inventors: Alexandra Barton-Sweeney, New Haven, CT (US); Honest Makamba, New Haven, CT (US)

(73) Assignee: Razzberry, Inc., New Haven, CT (US)

(21) Appl. No.: 15/752,364

(22) PCT Filed: Aug. 12, 2016
(86) PCT No.: PCT/US2016/046699

§ 371 Date: Feb. 13, 2018

Related U.S. Application Data
(60) Provisional application No. 62/205,380, filed on Aug. 14, 2015.
(60) Provisional application No. 62/254,402, filed on Nov. 12, 2015.
(60) Provisional application No. 62/290,501, filed on Feb. 3, 2016.
(60) Provisional application No. 62/322,273, filed on Apr. 14, 2016.

(51) Int. Cl. G01N 27/30; G01N 27/406; G01N 33/22

(52) U.S. Cl. CPC G01N 27/301; G01N 33/227; G01N 27/4062

(57) ABSTRACT

A sensor for detecting volatile materials comprising a working electrode having a surface; a reference electrode in electrical connection with the working electrode, the reference electrode comprising: a reference electrode surface; a nanocomposite coated on at least a portion of the reference electrode surface, the nanocomposite comprising: a compound of a metal used in the reference electrode, and nanoparticles, polymers, and proteins or a combination comprising at least one of the foregoing; and an ionic liquid in electrical connection with the reference electrode surface; wherein the electrical connection between the working electrode and the reference electrode is a solvent, or a conductive tape, or a solid polymer electrolyte; wherein when the sensor is exposed to an analyte, the sensor can generate an electrochemical signal; and wherein a surface of the working electrode or the conductive tape or the solid polymer electrolyte comprises carbon nanoparticles or a nanocomposite comprising carbon nanoparticles and noble metal nanoparticles is provided.

### CROSS-REFERENCE TO RELATED APPLICATIONS

This application claims priority to U.S. provisional patent application 62/205,380, filed Aug. 14, 2015; U.S. provisional patent application 62/254,402, filed Nov. 12, 2015; U.S. provisional patent application 62/290,501, filed Feb. 3, 2016; and U.S. provisional patent application 62/322,273, filed Apr. 14, 2016, the contents of each of which are hereby incorporated by reference in their entirety.

### BACKGROUND

One application of a stable solid state electrode in dry conditions is in the detection of explosive materials in air and other gaseous chemicals present in air. Explosive materials such as triacetone triperoxide (TATP) and hexmethylene triperoxide diamine (HMTD) can be made from over-the-counter chemicals, and have been used in terrorist attacks. Peroxide explosives such as TATP, HMTD, and other explosive materials such as pentaerythritol tetranitrate (PETN) are difficult to detect using optical techniques because they do not possess chromophores. Electrochemical detection is an alternative to optical detection because chromophores are not required for electrochemical detection. Electrochemical detection of explosives allows for portability of the sensors, which is not very easy with optical detection. This also allows for portable detectors for other gaseous chemicals that may be found in air for environmental monitoring.

There remains a need in the art for an electrochemical method to detect elusive peroxide-containing explosives materials such as TATP and HMTD and nitro-containing explosives such as PETN.

Additionally, conventional gas detectors often lack specificity. This results in a large number of false positives due to an inability to distinguish between the specific gas and other materials. There remains a need in the art for a highly-specific volatile substance sensor and improved portable volatile substance sensor devices.

### SUMMARY

Electrochemical detection using imprinting via solid state electrode sensor devices as described herein provides improved specificity, portability, and stability in the detection of volatile materials and gases, especially when used to detect chromophore-lacking explosive materials. The electrochemical sensing devices as described herein can also provide for miniaturized and inexpensive sensing devices.

A sensor for detecting explosive materials comprising: a working electrode having a surface; a reference electrode in electrical connection with the working electrode, the reference electrode comprising: a reference electrode surface; a compound of a metal used in the reference electrode coated on at least a portion of the reference electrode surface; a compound of a metal used in the reference electrode coated on at least a portion of the reference electrode surface, and an ionic liquid in electrical connection with the reference electrode surface; or a nanocomposite coated on at least a portion of the reference electrode surface, the nanocomposite comprising: a compound of a metal used in the reference electrode, and carbon nanoparticles, amyloid type nanofibrils, an adhesive protein, or a combination comprising at least one of the foregoing; wherein the electrical connection between the working electrode and the reference electrode is a solvent, or a conductive tape, or a gel such as a solid polymer electrolyte; wherein when the sensor is exposed to an analyte, the sensor can generate an electrochemical signal; and wherein a surface of the working electrode or the conductive tape or the solid polymer electrolyte comprises carbon nanoparticles or a nanocomposite comprising carbon nanoparticles and noble metal nanoparticles is provided.

A sensor for detecting explosive materials comprising: a working electrode having a surface; a reference electrode in electrical connection with the working electrode, the reference electrode comprising: a reference electrode surface; a nanocomposite coated on at least a portion of the reference electrode surface, the nanocomposite comprising: a compound of a metal used in the reference electrode, and carbon nanoparticles, amyloid type nanofibrils, an adhesive protein, or a combination comprising at least one of the foregoing; and an ionic liquid in electrical connection with the reference electrode surface; wherein the electrical connection between the working electrode and the reference electrode is a solvent, or a conductive tape, or a solid polymer electrolyte; wherein when the sensor is exposed to an analyte, the sensor can generate an electrochemical signal is provided.

A sensor for detecting explosive materials comprising: a working electrode having a surface comprising carbon nanoparticles or a nanocomposite comprising carbon nanoparticles and noble metal nanoparticles; a reference electrode in electrical connection with the working electrode, the reference electrode comprising: a reference electrode surface; a nanocomposite coated on at least a portion of the reference electrode surface, the nanocomposite comprising a compound of a metal used in the reference electrode, and carbon nanoparticles, amyloid type nanofibrils, an adhesive protein, PVB, or a combination comprising at least one of the foregoing; and an ionic liquid in electrical connection with the surface; wherein the electrical connection between the working electrode and the reference electrode is a solvent, or a conductive tape, or a solid polymer electrolyte; wherein when the sensor is exposed to an analyte, the sensor can generate an electrochemical signal is provided.

A sensor for detecting explosive materials comprising: a working electrode having a surface; a reference electrode in electrical connection with the working electrode, the reference electrode comprising: a reference electrode surface; a nanocomposite coated on at least a portion of the reference electrode surface, the nanocomposite comprising a compound of a metal used in the reference electrode, and carbon nanoparticles, amyloid type nanofibrils, an adhesive protein, PVB, or a combination comprising at least one of the foregoing; and an ionic liquid in electrical connection with the reference electrode surface; wherein the electrical connection between the working electrode and the reference electrode is a solvent, or a conductive tape, or the solid polymer electrolyte; wherein the conductive tape or solid polymer electrolyte comprises carbon nanoparticles or a nanocomposite comprising carbon nanoparticles and noble metal nanoparticles; wherein when the sensor is exposed to an analyte, the sensor can generate an electrochemical signal is provided.

A method of detecting explosive materials, comprising: providing a sensor as described herein; providing a sample of a target material to the sensor; measuring an electrochemical signal associated with the sensor; processing the electrical signal to generate an output that indicates the presence or absence of a material or the concentration level of a material that can turn into gaseous form is provided.

A method of detecting gaseous materials, comprising: providing a sensor as described herein; providing a sample of a target material to the sensor; measuring an electrochemical signal associated with the sensor; processing the electrical signal to generate an output that indicates the presence or absence of a material or the concentration level of a material that can turn into gaseous form is provided.

A method of detecting volatile materials, comprising: providing a sensor as described herein; providing a sample of a target material to the sensor; measuring an electrochemical signal associated with the sensor; processing the electrical signal to generate an output that indicates the presence or absence of a material or the concentration level of a material that can turn into gaseous form is provided.

A system for detecting an explosive material comprising a sensor as described herein is provided.

The reference electrode comprises a metal, the metal in the reference electrode can be gold, mercury, platinum, silver, palladium, copper, or a combination comprising at least one of the foregoing. A compound of a metal used in the reference electrode or other electrode can be an ionic or covalently bonded compound comprising the metal. In an embodiment, a compound of a metal used in the reference electrode is a salt, such as a chloride salt, an iodide salt, a sulfate salt, or other salt of the metal used in the reference electrode. In embodiments, a compound of a metal used in the reference electrode can be mercury chloride, silver chloride, silver iodide, copper sulfate, mercurous sulfate, or a combination comprising at least one of the foregoing. As an example, if the reference electrode comprises gold, a compound of a metal used in the reference electrode comprises gold, and the compound of a metal used in the reference electrode can be a salt of gold, such as sodium aurothiosulfate. The proteins can be any protein that exhibits strong binding characteristics, such as adhesive proteins, mussel proteins, fibrinogen, protofilaments, amyloid nanofibrils, or a combination comprising at least one of the foregoing. The polymers can include PVB (polyvinyl butyral) or any polymer that exhibits strong binding characteristics. The nanoparticles can be gold nanoparticles, silver nanoparticles, copper nanoparticles, zinc oxide nanoparticles, carbon nanoparticles, spherical carbon nanoparticles, fullerenes, quantum dots, graphene oxide, carbon nanotubes, nanofibers, carbon nanofibers, diamond nanoparticles, carbon quantum dots, titanium oxide nanoparticles, titanium dioxide (TiO2) nanoparticles, silicon oxide nanoparticles, gold nanoclusters, silver nanoclusters, europium oxide nanoparticles, iron oxide nanoparticles, diamond nanoparticles, graphene quantum dots, graphene nanoparticles, or a combination comprising at least one of the foregoing.

The above described and other features are exemplified by the following detailed description.

### DETAILED DESCRIPTION

Described herein is a solid state reference electrode, a sensor, methods of making the solid state electrode, and sensor, and methods of using the solid state electrode and sensor, for example, to detect explosive materials.

The solid state reference electrode and sensor may be used in the device described in commonly owned U.S. Nonprovisional application Ser. No. 14/662,411, filed Mar. 19, 2015, entitled “Health State Monitoring Device,” the contents of which are incorporated herein by reference in its entirety.

As used herein, “solid state electrode” means an electrode that does not contain liquid solutions or liquids in its structure.

The working electrode and reference electrode can be of any suitable size and shape. For example, the electrode can be a wire, a thin film on a surface, a pattern on a flexible substrate, a material, or ink. The electrode may be part of a printed sensor. The electrode can be any suitable thickness that allows the desired formation steps to occur and also allows fabrication into a desired device. The nanocomposite can be coated on substantially all or a portion of the surface of the reference electrode. For example, the electrode can be a generally two-dimensional shape, and the nanocomposite can be coated on one side, or a portion of one side of the electrode. Coating does not necessarily mean a uniform layer is formed. There may be holes, voids, or other areas where there is no nanocomposite or less nanocomposite or more nanocomposite than in other areas, as long as the nanocomposite coated surface performs in the desired manner and with the desired characteristics, as described herein. In an embodiment, the nanocomposite can comprise a compound of a metal used in an electrode, and also either nanoparticles, a protein or proteins, a polymer or polymers, or a combination comprising at least one of nanoparticles, a protein, and a polymer.

The carbon nanoparticles can be made from different sources. They can be made from sources such as amino acids, non-amino organic acids, alcohols, alkanes, monosaccharides, and biological materials. Specific sources include methane, ethanol, ethane, citric acid, gluconic acid, glucuronic acid, glucosamine, galactosamine, fructosamine, mannosamine and other carbon sources such as eggs. The carbon nanoparticles can be produced by heating the precursors, resulting in the loss of water molecules, and producing the carbon nanomaterial. The carbon nanoparticles can be of any suitable form, for example, carbon nanotubes (single-wall or multi-wall), graphene, fullerenes, diamond, carbon quantum dots, graphene quantum dots, or carbon nanofibers, or a combination comprising at least one of the foregoing. The carbon nanoparticles can be graphitic in structure, such as flat, disk-shaped, or irregularly shaped. Carbon nanoparticles can be fluorescent or non-fluorescent.

The carbon nanoparticles can be modified, for example, where a hydrophobic compound comprising an amine group and a thiol group is covalently bonded to the carbon nanoparticles. Further, the carbon nanoparticles can be modified with a hydrophobic compound containing a carboxylic group and a thiol. This modification can be carried out using conventional methods, such as carbodiimide coupling or Schiff base conjugation. The hydrophobic compound comprising an amine group and a thiol group can be any one of a number of compounds, such as 4-aminothiophenol or 5-amino-2-mercaptobenzimidazole. The hydrophobic compound comprising a carboxyl group and a thiol group can be 5-carboxy-2-mercaptobenzimidazole or compounds with a similar structure. The hydrophobic compound comprising an amine group and a thiol group can also include an aromatic group, which can reduce the solubility of the compound of the metal.

Hydrophobic compounds can include amine, thiol, aromatic, and carboxyl groups, such as 4-aminothiophenol, 5-amino-2-mercaptobenzimidazole, 5-carboxy-2-mercaptobenzimidazole, Thiophenol, 2-Napthalenethiol, and 9-Anthracenethiol.

The nanoparticles can have any suitable size and shape as long as the nanoparticles function in the desired methods and do not interfere with the operation of the solid state electrode. The nanoparticles are generally small, such that they may have an average diameter of less than or equal to 100 nanometers, in one embodiment less than or equal to 50 nanometers, in another embodiment less than or equal to 20 nanometers, in another embodiment less than or equal to 15 nanometers, and in still another embodiment less than or equal to 10 nanometers.

In an embodiment, a method of making a reference solid state electrode is provided, comprising: providing a metal electrode having a surface; attaching a nanocomposite comprising a compound of a metal used in the metal electrode, and nanoparticles, one or more proteins, and a polymer, or a combination comprising at least one of nanoparticles, onto at least a portion of the metal electrode surface. The nanocomposite can be attached to the surface of the metal electrode using either physical deposition or electrochemical deposition.

Physical deposition refers to any method, including chemical deposition, that does not use a voltage to attach the nanocomposite to the surface of the electrode. The compound of the metal used in the electrode can be produced by oxidation of the metal surface by using an oxidizing agent. The oxidizing agent can be washed away after the deposition of the layer of the compound of the metal used in the electrode or layer of the compound of the metal used in the electrode and nanocomposite. In one example, physical deposition comprises mixing the nanoparticles with the oxidizing agent in a solution, to form a composite solution, and applying the composite solution to the surface to produce a composite solid state electrode. The concentration of the oxidizing agent can be any suitable concentration to achieve the desired results, and can be 0.5 Molar (M)±0.25 M, and in one embodiment 0.1 M±0.05 M.

The oxidizing agent can be permanganate, dichromate, iron(III), perchlorate, periodate, hydrogen peroxide, chlorate, chromate, or iodate.

The nanocomposite can include a protein, one or more polymers, or nanoparticles, or a combination comprising at least one of the foregoing, and the protein, one or more polymers, or nanoparticles, or combination, can be mixed with the oxidizing agent prior to attaching the nanocomposite onto the surface.

Electrochemical deposition uses a voltage to attach the nanocomposite to the surface of the electrode. Electrochemical deposition can include applying a voltage or current to the surface in an acid solution, forming a surface coated with a compound of a metal used in the electrode; and electrochemically depositing the nanocomposite onto the surface coated with a compound of a metal used in the electrode. In one example, electrochemically depositing includes applying a voltage or current to the surface, forming a nanoparticle-compound of a metal used in the electrode composite coated surface. As an example, the electrochemical deposition can be performed by applying 20 μA for 1 minute or 2 minutes. The acid solution can be sulphuric acid solution, nitric acid solution, potassium chloride, acidified potassium chloride, potassium chloride acidified with hydrochloric acid, hydrochloric acid solution, or phosphoric acid.

The working electrode, reference electrode, and other components of the sensor can be deposited on a substrate by techniques such as screen printing, roll-to-roll printing, aerosol deposition, inkjet printing, thin film deposition, or electroplating. The substrate can be a flexible or rigid polymer, a textile, a mat, glass, metal substrate, or other printed material. The substrate can be non-electrically conductive. The substrate can be electrically conductive. There may be intermediate layers between the substrate and electrodes.

Special equipment may be made to build the sensors and their components, such as the working solid state electrodes and reference electrodes, according to the description herein.

The analyte measurements can be used alone or in combination with other analytes, markers, physiological data, environmental data, user data or population data, and pattern recognition/informatics, to determine the state, or health state of the organism or environment being measured. The changes in the solid state electrode measurements can be monitored by an algorithm using either the exact measurement levels, or their values in relation to other measurements of the same sensor, or in relation to measurements of other markers, or sensors, or data such as population data or user data. The drift or change in sensor or solid state electrode measurements over time, or the accumulation of data points over time, can be analyzed to assist in determination of the analyte concentration. Z scores, normalization, and other data modeling approaches can be used in the analysis. This information can be used either to show the levels of the analyte or to be used to recognize the existence of, state of, or tendency toward a condition. The measurements of the electrodes themselves that make up a sensor or multiple sensors, can be compared to each other to assist in analysis. The differences between electrodes, such as the differential between the working and reference electrodes, can be used to determine an analyte level. In one case, one of the electrodes is grounded out in relation to the other electrode, to obtain a single differential value for comparison. In other cases, electrode measurements may be used separately in the analysis. Additional electrodes may be used to help in analyzing the level of the analyte, by providing an additional measurement value of the same analyte; selecting for another analyte; or for use in detecting other influences that may impact the measurements of the electrodes in general. For example, what would normally be a two-electrode system may utilize additional electrodes, such as a counter electrode to monitor fluctuations in current. Further, the counter electrode can help in drawing the current away from the reference electrode, which helps in conserving the composition of the reference electrode. The data model can be used with a group of these inputs where some inputs may influence the analysis of other inputs in order to approximate the chemical level (e.g. temperature influencing chemical levels, fluctuations in current influencing chemical levels).

The data from the solid state electrodes and sensor can be collected and analyzed via an electronic device or sent over a network to an external device, such as a mobile phone, for analysis, or manually inputted into software for analysis.

While many of the examples involve a two-electrode system (working and reference solid state electrodes), this is for exemplary purposes and the claims should not be so limited, and other embodiments may include an electrode system that uses any number of electrodes. For example, the sensor can have a working electrode, a reference electrode, and a counter electrode. In an embodiment, the electrode system comprises multiple electrochemical cells with one or more than one, such as 1 to 5, or 1 to 15, or 1 to 25 electrodes for each cell. The electrode system can have multiple electrochemical cells that share electrodes. In an embodiment, the electrode system can have an array of electrochemical cells each with their own working electrodes, and a shared reference electrode wherein the cells can sense separate analytes in the same sample that is in contact with the cells and electrodes.

The electrodes, methods, and sensors are further illustrated by the following non-limiting examples.

### EXAMPLES

### Physical Deposition

The general steps in one embodiment of a physical deposition process to prepare a solid state electrode include: deposition of nanoparticles with a compound of a metal used in the electrode onto the metal electrode surface to make the solid state electrode; chemically modifying the nanoparticles to reduce the surface charge, then depositing the nanoparticles together with the compound of the metal used in the electrode onto the metal electrode surface to make the solid state electrode; and attachment of proteins, such as strongly adhesive proteins, one or more polymers, such as amyloid type nanofibrils, or PVB, to act as a diffusion barrier. In some cases, nanocomposites of proteins, and nanoparticles, such as titanium dioxide (TiO2) nanoparticles, can be used to protect the solid state electrode. Because some sample mediums or device setups can be abrasive to the sensor, such as soil samples or where the sensor is exposed to friction or placed in direct contact with an external surface, it may be desirable to protect the solid state electrode with “tough” nanocomposites of proteins and nanoparticles. Without protection, the soil particles or friction, for example, can erode the solid state electrodes. Solid state electrode erosion means that the device would not be as durable as a solid state electrode that did not erode. Protection of the solid state electrode can also be used for other analytes that may contain particles that can damage the electrode, including drug suspensions and environmental water samples, for example.

Another embodiment for attaching nanoparticles to make stable solid state reference electrodes involves using modified nanoparticles. In this approach, the nanoparticles are first modified with hydrophobic compounds containing an amine group and a thiol group via covalent bonding. The nanoparticles can also be modified with a hydrophobic compound containing a carboxyl group and a thiol group. Thiol containing compounds are known to interact strongly with metal atoms. The surface modification of the nanoparticles reduces the surface charge. The thiol containing compounds can be attached to the nanoparticles using the amine group by well-known chemical reactions. The modified nanoparticles can be attached to the compound of the metal using physical deposition in combination with an oxidizing agent. The modified nanoparticles interact strongly with the metal atoms in the compound of the metal via the thiol groups, creating a robust structure. Further, these nanoparticles reduce the solubility of the compound of the metal. The reduction in the solubility of the compound of the metal slows down the loss of it from the reference solid state electrode.

Another embodiment to stabilize the solid state electrode is attachment of proteins, and/or polymers to the electrodes. The proteins are mixed with an oxidizing agent and nanoparticles and physically attached to the metal electrode to produce composites that strongly stick to the surface. These proteins are also used as thin layers on top of electrodes modified as described above. In some cases, an oxidant can be used to accelerate the crosslinking of the proteins. In some cases, it is important for the proteins to be cross-linked so that they can effectively encapsulate the electrode. Crosslinking is believed to impart physical stability to the protein. Polymers or peptides can be mixed with an oxidizing agent and nanoparticles in the same way as the proteins to produce nanocomposite reference solid state electrodes. A top layer of polymers can also be deposited on top of the electrode to act as a diffusion barrier, similarly to the proteins.

Polymers, including those which have been shown to adhere strongly to surfaces, such as polyvinyl butyral (PVB) can be used.

The following proteins can be used, which have been shown to adhere strongly to surfaces, such as amyloid fibrils, amyloid nanofibrils, adhesive proteins, fibrinogen, protofilaments, or mussel proteins. Unless otherwise indicated, “strongly binding” means binds sufficiently to allow the desired interactions to occur, or for the desired functions to occur, as described herein.

### Electrochemical Deposition

The general steps in an electrochemical deposition process to prepare a solid state electrode include: electrochemical deposition of nanoparticles with a compound of a metal used in the electrode onto the electrode surface, chemically modifying the nanoparticles to reduce the surface charge, then electrochemically depositing the nanoparticles together with the compound of a metal used in the electrode onto the electrode surface; and electrochemical attachment of proteins and/or polymers to act as a diffusion barrier.

For electrochemical deposition of compound of a metal used in the electrode onto the metal surface of the electrode, 1 M or 2 M, for example, of an acid is used and a voltage or current applied. As an example a current of 20 μA for 1 minute or 2 minutes is applied. The applied voltage produces a coating of a compound of a metal used in the electrode on the metal surface. Electrochemical deposition of nanoparticles from solution is performed the deposited compound of the metal used in the electrode layer. The nanoparticles attach to the compound of the metal layer via redox processes. A portion of the surface of the compound of a metal used in the electrode, or the entire surface of the compound of a metal used in the electrode can be coated with nanoparticles, depending on the amount of component in solution, the charge, and other factors known in the art.

Besides covering the layer of the compound of a metal used in the electrode with nanoparticles, mixed composites of the compound of a metal used in the electrode with nanoparticles can be made. An acid is mixed with nanoparticles in a solution, and the mixed solution is applied to the electrode. Next, a potential is applied to the electrode to electrochemically attach the compound of a metal used in the electrode-nanoparticles composites to the electrode surface. Because of the electrochemical processes, the compound of a metal used in the electrode and the nanoparticles are chemically bound which produces a robust electrode.

Nanoparticles that are first chemically modified then deposited together with the compound of the metal used in the electrode, can be used, as described above. Hydrophobic compounds containing an anime group, a thiol group, and an aromatic group are used in an example. Next, the modified nanoparticles are mixed with acid to form a solution. The solution mixture is applied to the electrode surface and a potential is applied to the electrode. The application of the potential electrochemically deposits the nanoparticles together with the compound of a metal used in the electrode on the electrode surface. The thiol modified nanoparticles form strong bonds with the metal atoms in the compound of a metal used in the electrode during the electrochemical process. The strong bonding produces a stable structure. Further, the aromatic hydrophobic groups attached to the nanoparticles reduce the solubility of the compound of the metal used in the electrode. Although Applicant does not wish to be bound by any theory provided, reducing the solubility of the compound of a metal used in the electrode is known to be useful in slowing down its loss from the electrode during operation.

Similarly to the physical deposition methods, proteins can be attached to the electrode prepared using electrochemical methods. Electrodes prepared using the approaches above are modified on the surface using the proteins. A small drop of a protein solution, in many cases the solution is water, is applied to the electrodes. Next, a drop of oxidant is added to the electrodes. Depending on the type of protein, the electrodes are left for 30 minutes or overnight, for example, to allow the oxidant to crosslink the protein. The crosslinked protein layer on top of the electrode reduces loss of the compound of the metal.

Polymers such as PVB can be attached to the electrode by drop casting a solution of PVB in organic solvent.

In a particular example, electrochemical deposition of a compound of a metal used in the electrode in the presence of the protein is performed. The protein is mixed with acid and the solution placed on the electrode. Application of voltage or current deposits the compound of a metal used in the electrode together with the protein. Another embodiment is to mix the protein with nanoparticles, and acid. Another embodiment is to mix the protein with nanoparticles. Applying a voltage deposits a nanocomposite of a compound of a metal used in the electrode, nanoparticles, and one or more proteins.

### Surface Analysis

The solid state electrodes produced by the methods outlined above are characterized using microscopic imaging and spectroscopy. Microscopic techniques for imaging include atomic force microscopy (AFM) and scanning electron microscopy (SEM). AFM provides images of structures that are as small as 2 nanometers. The detail in AFM imaging provides information on the very small nanocomposites that are produced during the deposition. On the other hand, SEM provides information on the micro-sized structures that are produced and on the distribution of the nanostructures. Spectroscopy is used to identify the chemical groups that are on the surface of the solid state electrode. While the materials that are attached to the electrode are known, chemical changes may occur during the deposition, resulting in the alteration of the chemistry of these materials. Spectroscopic techniques include infra-red spectroscopy (IR), x-ray photoelectron spectroscopy (XPS), Raman spectroscopy, and fluorescence spectroscopy.

### Explosives Detection

Sensing Using Modified Solid State Working Electrode

The solid state reference electrodes described here can be used in the electrochemical detection of explosives. In an embodiment, the solid state reference electrodes can be made from noble metals, such as gold, platinum, silver, palladium, mercury, or copper, for the electrochemical detection of explosives. In embodiment, the solid state reference electrode can be unmodified and only comprise a metal electrode and a compound of the metal used in the electrode. In an embodiment, the working electrodes can be made from noble metals or carbon. In an embodiment, the working solid state electrode is modified with nanocomposites that can help capture the explosive material by increasing the surface area of the working electrode, which allows for absorbing more material for detection. In an embodiment, the synthesis of the solid state reference electrode includes an ionic liquid that enhances the conductivity in the solid state reference electrode. Examples of ionic liquids that can be used in the solid state reference electrode include 1-ethyl-3-methylimidazolium bis(trifluoromethylsulfonyl)imide ([EMIM][N(Tf)₂]) and 1-butyl-3-methylimidazolium trifluoromethanesulfonate [BMIM][CF₃SO₃]. In an embodiment, the working electrode is electrochemically modified with carbon nanoparticles, or with carbon nanoparticles in nanocomposites with noble metal nanoparticles or metal oxide nanoparticles. The carbon nanoparticles can include carbon nanotubes (single-wall or multi-wall), graphene, graphene oxide, fullerenes, diamond, carbon quantum dots, graphene quantum dots, carbon nanofibers, spherical carbon nanoparticles, or a combination comprising at least one of the foregoing. The metal nanoparticles can include gold, silver, platinum, or palladium, at a concentration of between 0.01 mM and 0.1 mM, for example. The metal oxide nanoparticles can include titanium oxide, zinc oxide, silicone oxide, europium oxide, or iron oxide. The ionic liquid can be physically adsorbed in the organic solvent, in an embodiment.

An organic solvent, such as acetonitrile, methanol, or ethanol is applied so that it covers the working electrode and the reference electrode. In an embodiment, the device is then exposed to the explosives from the air and the organic solvent dissolves the explosives, resulting in a change in the potential between the reference electrode and the working electrode. These solvents are compatible with the explosives such as TATP and HMTD. In this configuration, the sensing is based on the change in potential when the explosive is dissolved in the solvent when compared to the solvent without explosives.

In another embodiment, the organic solvent is replaced with a conductive tape that provides electrical contact between the solid state reference electrode and the solid state working electrode. The conductive tape is placed between the solid state reference electrode and the solid state working electrode, so that the conductive tape touches both electrodes. The conductive tape allows for the sensing of the explosive materials from the air or other dry conditions. When the explosive materials come into contact with the working electrode there is a change in potential which is detected. Conductive tapes are readily available.

In another embodiment, solid polymer electrolytes (SPE) are used. SPE are conductive solid materials that are made of polymers mixed with ionic liquids or polymers synthesized in-situ in the presence of an ionic liquid. SPE provide an alternative to conductive tapes in the sensing of explosives in the air in dry conditions. Conductive ion gels are a type of SPE that can be used for dry sensing of explosives from the air.

In an embodiment, the sensor can be simply waved in the sample, such as air or placed in contact with the sample, such as air, to contact the sensor with the potential source of explosives. In an embodiment, pumps can be used to pull the sample, such as air, into a sampling chamber. In an embodiment, the sampling chamber can include a membrane or absorbent paper that is contacted with a solvent, as discussed elsewhere herein. In an embodiment, the conductive ion gels are deposited on top of the two solid state electrodes using acrylic photo-polymerization in which ionic liquids and conductive salts are also included. In an embodiment, acrylic monomers are mixed with ionic liquid, a conductive salt and photo-initiator and dissolved in organic solvent. The solution mixture is placed on top of the electrodes and polymerization of the solution mixture is achieved by exposing the solution mixture to UV light for a specified time, during which a gel is formed. Molecular imprinting of the explosives in the gels is done to enhance sensitivity and selectivity. Molecular imprinting describes generally when the molecular structure of a substance, such as the explosive, is imprinted in the gel. The explosives are included in the solution mixture as described above and patterns (imprints) of the explosives are left in the mixture when the solid polymer is produced. The explosive is then washed away from the solid polymer after the polymerization, leaving the imprints in the solid polymer material. Similarly, the substance, such as the explosive, can be imprinted in proteins by mixing the substance with the proteins, such as amyloid type nanofibrils, or by mixing the substance with the amyloid proteins, and allowing the proteins to self-assemble to produce imprints in the protein fibrils. The explosive is then washed away after self-assembly of the protein fibrils, such as the amyloid fibrils. The washing process also removes other remaining chemicals that are left behind after polymerization. Sensing using this design is the same as with the conductive tape. For this technology, molecular imprinting can be applied to any substance that can be imprinted in a polymer network, and that easily turns into gaseous form. Further, the substance should easily diffuse in air in molecular form for detection to occur when sampling air. Examples of such substances include the explosives TATP and HMTD, and organic compounds such as benzene, pthalates, mycotoxins, tetrahydrofuran, acetone, alkanes, alcohols and toluene. The device is exposed to the material, and when the material molecules come into contact with the working electrode, the potential between the solid state reference electrode and the solid state working electrode changes. The sensing can also be performed using a three electrode arrangement. In a three electrode system, the material, for example from the air, can be detected by measuring the impedance of current flowing between the working electrode and the counter electrode. An impedance analyzer can also be used. In addition to being hand-held, these devices for electronic detection of the materials described herein, such as explosives, in dry conditions can be placed at various points in public buildings to provide quick alerts on the presence of the materials. Further, these devices can be used on aircraft that do air sampling for gaseous chemicals either in the exterior or interior environments.

### Sensing Using Unmodified Working Electrode

A reference electrode which includes ionic liquids can be used with an unmodified working microelectrode. This embodiment can also be used for sensing explosives from the air in dry conditions. With the unmodified electrode, a conductive tape or a conductive gel can be modified with nanoparticles.

In an embodiment, the reference electrode can be used without an ionic liquid if conductivity does not need to be improved.

In an embodiment, the conductive tape is modified by soaking it in a dilute solution of carbon nanoparticles or a mixture of carbon nanoparticles with metal nanoparticles to form nanocomposites. After soaking, the conductive tape is dried and placed on top of the microelectrodes to provide electrical contact between the microelectrodes. The sensing is done by exposing the device to the material to be detected, such as the explosive. The material is absorbed by the nanocomposites on the conductive tape, resulting in a change of potential between the reference electrode and the working electrode.

The SPE (conductive gel) is attached as described above using photo-polymerization or thermal-polymerization. Before the polymerization, carbon nanoparticles and/or metal nanoparticles are mixed with the other components. After the nanomaterials are mixed with the other components, the mixture is placed on top of the electrodes and polymerization is performed by exposing to UV light or heat. This results in a nanocomposite conductive ion gel. Sensing is based on the interaction between the nanocomposite ion gel and the material, such as the explosive, from the air, which results in a change of potential between the reference electrode and the working electrode. As with the modified working electrode, the sensing can also be done using a three electrode arrangement. In a three electrode system, the explosive from the air is detected by measuring the impedance of current flowing between the working electrode and the counter electrode, while the reference electrode helps to stabilize the potential.

The compositions, methods, articles, and other aspects are further described by the Embodiments below.

Embodiment 1: A sensor for detecting volatile or semi-volatile materials comprising: a working electrode having a surface; a reference electrode in electrical connection with the working electrode, the reference electrode comprising: a reference electrode surface; a compound of a metal used in the reference electrode coated on at least a portion of the reference electrode surface; a compound of a metal used in the reference electrode coated on at least a portion of the reference electrode surface, and an ionic liquid in electrical connection with the reference electrode surface; or a nanocomposite coated on at least a portion of the reference electrode surface, the nanocomposite comprising: a compound of a metal used in the reference electrode, and carbon nanoparticles, a strongly binding protein, a strongly binding polymer, or a combination comprising at least one of the foregoing; wherein the electrical connection between the working electrode and the reference electrode is a solvent, or a conductive tape, or a solid polymer electrolyte; wherein when the sensor is exposed to an analyte, the sensor can generate an electrochemical signal; and wherein a surface of the working electrode or the conductive tape or the solid polymer electrolyte comprises carbon nanoparticles or a nanocomposite comprising carbon nanoparticles and noble metal nanoparticles.

Embodiment 2: A sensor for detecting volatile or semi-volatile materials comprising: a working electrode having a surface; a reference electrode in electrical connection with the working electrode, the reference electrode comprising: a reference electrode surface; a nanocomposite coated on at least a portion of the reference electrode surface, the nanocomposite comprising: a compound of a metal used in the reference electrode, and carbon nanoparticles, a strongly binding protein, a strongly binding polymer, or a combination comprising at least one of the foregoing; and an ionic liquid in electrical connection with the reference electrode surface; wherein the electrical connection between the working electrode and the reference electrode is a solvent, or a conductive tape, or a solid polymer electrolyte; wherein when the sensor is exposed to an analyte, the sensor can generate an electrochemical signal.

Embodiment 3: The sensor of Embodiment 1 or 2, wherein a surface of the working electrode or the conductive tape or the solid polymer electrolyte comprises carbon nanoparticles or a nanocomposite comprising carbon nanoparticles and noble metal nanoparticles.

Embodiment 4: The sensor of Embodiment 1 or 2, wherein the solid polymer electrolyte is modified via molecular imprinting to detect a material.

Embodiment 5: The sensor of Embodiment 1 or 2, wherein the working electrode and reference electrode is each independently a noble metal, preferably silver, gold, platinum, palladium, copper, or carbon, or a combination comprising at least one of the foregoing.

Embodiment 6: The sensor of Embodiment 1 or 2, wherein the working electrode and reference electrode is each independently silver, gold, platinum, palladium, copper, or carbon, or a combination comprising at least one of the foregoing.

Embodiment 7: The sensor of Embodiment 1 or 2, wherein the compound of a metal is mercury chloride, silver chloride, silver iodide, copper sulfate, mercurous sulfate, or a combination comprising at least one of the foregoing.

Embodiment 8: A sensor for detecting volatile or semi-volatile materials comprising: a working electrode having a surface comprising carbon nanoparticles or a nanocomposite comprising carbon nanoparticles and noble metal nanoparticles; a reference electrode in electrical connection with the working electrode, the reference electrode comprising: a reference electrode surface; a nanocomposite coated on at least a portion of the reference electrode surface, the nanocomposite comprising a compound of a metal used in the reference electrode, and carbon nanoparticles, a strongly binding protein, a strongly binding polymer, or a combination comprising at least one of the foregoing; and an ionic liquid in electrical connection with the surface; wherein the electrical connection between the working electrode and the reference electrode is a solvent, or a conductive tape, or a solid polymer electrolyte; wherein when the sensor is exposed to an analyte, the sensor can generate an electrochemical signal.

Embodiment 9: The sensor of Embodiment 8, wherein the solid polymer electrolyte is modified via molecular imprinting to detect a material.

Embodiment 10: The sensor of Embodiment 8, wherein the working electrode and reference electrode is each independently a noble metal, preferably silver, gold, platinum, palladium, copper, or carbon, or a combination comprising at least one of the foregoing.

Embodiment 11: The sensor of Embodiment 8, wherein the compound of a metal used in the reference electrode is mercury chloride, silver chloride, silver iodide, copper sulfate, mercurous sulfate, or a combination comprising at least one of the foregoing.

Embodiment 12: A sensor for detecting volatile or semi-volatile materials comprising: a working electrode having a surface; a reference electrode in electrical connection with the working electrode, the reference electrode comprising: a reference electrode surface; a nanocomposite coated on at least a portion of the reference electrode surface, the nanocomposite comprising a compound of a metal used in the reference electrode, and carbon nanoparticles, a strongly binding protein, a strongly binding polymer, or a combination comprising at least one of the foregoing; and an ionic liquid in electrical connection with the reference electrode surface; wherein the electrical connection between the working electrode and the reference electrode is a solvent, or a conductive tape, or the solid polymer electrolyte; wherein the conductive tape or solid polymer electrolyte comprises carbon nanoparticles or a nanocomposite comprising carbon nanoparticles and noble metal nanoparticles; wherein when the sensor is exposed to an analyte, the sensor can generate an electrochemical signal.

Embodiment 13: The sensor of Embodiment 12, wherein the solid polymer electrolyte is modified via molecular imprinting to detect a material.

Embodiment 14: The sensor of Embodiment 12, wherein the working electrode and reference electrode are each independently a noble metal, preferably silver, gold, platinum, palladium, copper, or carbon, or a combination comprising at least one of the foregoing.

Embodiment 15: The sensor of Embodiment 12, wherein the compound of a metal used in the reference electrode is mercury chloride, silver chloride, silver iodide, copper sulfate, mercurous sulfate, or a combination comprising at least one of the foregoing.

Embodiment 16: The sensor of any one or more of Embodiments 1 to 15, wherein the signal indicates the presence or absence of an explosive material.

Embodiment 17: The sensor of any one or more of Embodiments 1 to 15, wherein the signal indicates the presence or absence of a compound that can turn into gaseous form.

Embodiment 18: The sensor of any one or more of Embodiments 1 to 15, wherein the analyte is a peroxide-containing explosive material, preferably TATP or HMTD, a nitro-containing explosive material, preferably PETN, or organic compound that can turn into gaseous form; or organic compound such as benzene, pthalates, mycotoxins, tetrahydrofuran, acetone, alkanes, alcohols and toluene.

Embodiment 19: The sensor of any one or more of Embodiments 1 to 15, wherein the carbon nanoparticles are single-wall or multi-wall carbon nanotubes, graphene, fullerenes, diamond, carbon quantum dots, or carbon nanofibers, or a combination comprising at least one of the foregoing.

Embodiment 20: The sensor of any one or more of Embodiments 1 to 15, further comprising a counterelectrode.

Embodiment 21: The sensor of any one or more of Embodiments 1 to 15, wherein the explosive materials are detected in air, on a conductive absorbent material, or in an organic liquid.

Embodiment 22: The sensor of any one or more of Embodiments 1 to 15, wherein the compound is detected in air, on a conductive absorbent material, or in an organic liquid.

Embodiment 23: The sensor of any one or more of Embodiments 1 to 15, wherein the solid polymer electrolyte is modified to detect a substance via molecular imprinting.

Embodiment 24: A method of detecting volatile or semi-volatile materials, comprising:

providing a sensor of any one or more of Embodiments 1 to 23; providing a sample of a target material to the sensor; measuring an electrochemical signal associated with the sensor; processing the electrical signal to generate an output that indicates the presence or absence of a material that can turn into gaseous form.

Embodiment 25: The method of Embodiment 24, wherein the material is an explosive material.

Embodiment 26: The method of Embodiment 24, wherein the material is an organic compound.

Embodiment 27: The method of Embodiment 26, wherein the explosive material is peroxide-containing or nitrate-containing.

Embodiment 28: The method of Embodiment 25, wherein the explosive material is TATP, HMTD, or PETN.

Embodiment 29: The method of claim 26, wherein the organic compound is benzene, pthalates, mycotoxins, tetrahydrofuran, acetone, alkanes, alcohols or toluene.

Embodiment 30: A system for detecting an explosive material comprising the sensor of any one or more of claims 1 to 23.

Embodiment 31: A system for detecting a material that can turn into gaseous form comprising the sensor of any one or more of claims 1 to 23.

Embodiment 32: The sensor of any one or more of Embodiments 1 to 15, wherein the signal indicates the presence or absence of a substance that can be imprinted in a polymer network.

Embodiment 33: The sensor of any one or more of Embodiments 1 to 15, wherein the analyte is a peroxide-containing explosive material, such as TATP or HMTD, a nitro-containing explosive material, such as PETN, or organic compound, such as benzene, pthalates, mycotoxins, tetrahydrofuran, acetone, alkanes, alcohols and toluene.

Embodiment 34: The method of Embodiment 24, wherein the material is an organic compound that can turn into a gaseous form, such as benzene, pthalates, mycotoxins, tetrahydrofuran, acetone, alkanes, alcohols and toluene.

Embodiment 35: The method of Embodiment 24, wherein the material is an organic liquid, such as benzene, tetrahydrofuran, acetone, alkanes, alcohols and toluene.

Embodiment 36: The sensor of any one or more of Embodiments 1 to 23 or 32 to 33, wherein multiple sensors share a reference electrode.

Embodiment 37: The sensor of any one or more of Embodiments 1 to 23 or 32 to 33, wherein the strongly binding polymer is PVB (polyvinyl butyral).

Embodiment 38: The sensor of any one or more of Embodiments 1 to 23 or 32 to 33, wherein the strongly binding protein is an adhesive proteins, a mussel protein, a fibrinogen, a protofilament, amyloid fibrils, amyloid nanofibrils, or a combination comprising at least one of the foregoing.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention.

All ranges disclosed herein are inclusive of the endpoints, and the endpoints are independently combinable with each other. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or” unless clearly stated otherwise. It is to be understood that the described elements may be combined in any suitable manner in the various embodiments.

1. A sensor for detecting volatile or semi-volatile materials comprising:
a working electrode having a surface;
a reference electrode in electrical connection with the working electrode, the reference electrode comprising:
a reference electrode surface;
a compound of a metal used in the reference electrode coated on at least a portion of the reference electrode surface;
a compound of a metal used in the reference electrode coated on at least a portion of the reference electrode surface, and an ionic liquid in electrical connection with the reference electrode surface; or
a nanocomposite coated on at least a portion of the reference electrode surface, the nanocomposite comprising: a compound of a metal used in the reference electrode, and carbon nanoparticles, a strongly binding protein, a strongly binding polymer, or a combination comprising at least one of the foregoing;
wherein the electrical connection between the working electrode and the reference electrode is a solvent, or a conductive tape, or a solid polymer electrolyte;
wherein when the sensor is exposed to an analyte, the sensor can generate an electrochemical signal; and
wherein a surface of the working electrode or the conductive tape or the solid polymer electrolyte optionally comprises carbon nanoparticles or a nanocomposite comprising carbon nanoparticles and noble metal nanoparticles.

2. A sensor for detecting volatile or semi-volatile materials comprising:
a working electrode having a surface;
a reference electrode in electrical connection with the working electrode, the reference electrode comprising:
a reference electrode surface;
a nanocomposite coated on at least a portion of the reference electrode surface, the nanocomposite comprising:
a compound of a metal used in the reference electrode, and carbon nanoparticles, a strongly binding protein, a strongly binding polymer, or a combination comprising at least one of the foregoing; and
an ionic liquid in electrical connection with the reference electrode surface;
wherein the electrical connection between the working electrode and the reference electrode is a solvent, or a conductive tape, or a solid polymer electrolyte;
wherein when the sensor is exposed to an analyte, the sensor can generate an electrochemical signal.

3. The sensor of claim 1, wherein a surface of the working electrode or the conductive tape or the solid polymer electrolyte comprises carbon nanoparticles or a nanocomposite comprising carbon nanoparticles and noble metal nanoparticles.

4. The sensor of claim 1, wherein the solid polymer electrolyte is modified via molecular imprinting to detect a material.

5. The sensor of claim 1, wherein the working electrode and reference electrode is each independently a noble metal, preferably silver, gold, platinum, palladium, copper, or carbon, or a combination comprising at least one of the foregoing.

6. The sensor of claim 1, wherein the working electrode and reference electrode is each independently silver, gold, platinum, palladium, copper, or carbon, or a combination comprising at least one of the foregoing.

7. The sensor of claim 1, wherein the compound of a metal is mercury chloride, silver chloride, silver iodide, copper sulfate, mercurous sulfate, or a combination comprising at least one of the foregoing.

8. The sensor of claim 1, wherein the strongly binding polymer is PVB (polyvinyl butyral).

9. The sensor of claim 1, wherein the protein is an adhesive protein, a mussel protein, a fibrinogen, a protofilament, amyloid fibrils, amyloid nanofibrils, or a combination comprising at least one of the foregoing.

10. A sensor for detecting volatile or semi-volatile materials comprising:
a working electrode having a surface comprising carbon nanoparticles or a nanocomposite comprising carbon nanoparticles and noble metal nanoparticles;
a reference electrode in electrical connection with the working electrode, the reference electrode comprising:
a reference electrode surface;
a nanocomposite coated on at least a portion of the reference electrode surface, the nanocomposite comprising
a compound of a metal used in the reference electrode, and
carbon nanoparticles, a strongly binding protein, a strongly binding polymer, or a combination comprising at least one of the foregoing; and
an ionic liquid in electrical connection with the surface;
wherein the electrical connection between the working electrode and the reference electrode is a solvent, or a conductive tape, or a solid polymer electrolyte;
wherein when the sensor is exposed to an analyte, the sensor can generate an electrochemical signal.

11. The sensor of claim 10, wherein the solid polymer electrolyte is modified via molecular imprinting to detect a material.

12. The sensor of claim 10, wherein the working electrode and reference electrode is each independently a noble metal, preferably silver, gold, platinum, palladium, copper, or carbon, or a combination comprising at least one of the foregoing.

13. The sensor of claim 10, wherein the compound of a metal used in the reference electrode is mercury chloride, silver chloride, silver iodide, copper sulfate, mercurous sulfate, or a combination comprising at least one of the foregoing.

14. The sensor of claim 10, wherein the strongly binding polymer is PVB (polyvinyl butyral).

15. The sensor of claim 10, wherein the protein is an adhesive protein, a mussel protein, a fibrinogen, a protofilament, amyloid fibrils, amyloid nanofibrils, or a combination comprising at least one of the foregoing.

16. A sensor for detecting explosive materials comprising:
a working electrode having a surface;
a reference electrode in electrical connection with the working electrode, the reference electrode comprising:
a reference electrode surface;
a nanocomposite coated on at least a portion of the reference electrode surface, the nanocomposite comprising
a compound of a metal used in the reference electrode, and carbon nanoparticles, a strongly binding protein, a strongly binding polymer, or a combination comprising at least one of the foregoing; and
an ionic liquid in electrical connection with the reference electrode surface;
wherein the electrical connection between the working electrode and the reference electrode is a solvent, or a conductive tape, or the solid polymer electrolyte; wherein the conductive tape or solid polymer electrolyte comprises carbon nanoparticles or a nanocomposite comprising carbon nanoparticles and noble metal nanoparticles;
wherein when the sensor is exposed to an analyte, the sensor can generate an electrochemical signal.

17. The sensor of claim 16, wherein the solid polymer electrolyte is modified via molecular imprinting to detect a material.

18. The sensor of claim 16, wherein the working electrode and reference electrode are each independently a noble metal, preferably silver, gold, platinum, palladium, copper, or carbon, or a combination comprising at least one of the foregoing.

19. The sensor of claim 16, wherein the compound of a metal used in the reference electrode is mercury chloride, silver chloride, silver iodide, copper sulfate, mercurous sulfate, or a combination comprising at least one of the foregoing.

20. The sensor of claim 16, wherein the strongly binding polymer is PVB (polyvinyl butyral).

21. The sensor of claim 16, wherein the protein is an adhesive proteins, a mussel protein, a fibrinogen, a protofilament, amyloid fibrils, amyloid nanofibrils, or a combination comprising at least one of the foregoing.

22. The sensor of claim 1, wherein the signal indicates the presence or absence of an explosive material.

23. The sensor of claim 1, wherein the signal indicates the presence or absence of a compound that can turn into gaseous form.

24. The sensor of claim 1, wherein the analyte is a peroxide-containing explosive material, preferably TATP or HMTD, a nitro-containing explosive material, preferably PETN, or organic compound that can turn into gaseous form; or organic compound such as benzene, pthalates, mycotoxins, tetrahydrofuran, acetone, alkanes, alcohols and toluene.

25. The sensor of claim 1, wherein the carbon nanoparticles are single-wall or multi-wall carbon nanotubes, graphene, fullerenes, diamond, carbon quantum dots or carbon nanofibers, or a combination comprising at least one of the foregoing.

26. The sensor of claim 1, further comprising a counterelectrode.

27. The sensor of claim 16, wherein the explosive materials are detected in air, on a conductive absorbent material, or in an organic liquid.

28. The sensor of claim 1, wherein the analyte is detected in air, on a conductive absorbent material, or in an organic liquid.

29. The sensor of claim 1, wherein the solid polymer electrolyte is modified to detect a substance via molecular imprinting.

30. A method of detecting volatile or semi-volatile materials, comprising:
providing a sensor of claim 1;
providing a sample of a target material to the sensor;
measuring an electrochemical signal associated with the sensor;
processing the electrical signal to generate an output that indicates the presence or absence of a material that can turn into gaseous form.

31. The method of claim 30, wherein the material is an explosive material.

32. The method of claim 30, wherein the material is an organic compound.

33. The method of claim 30, wherein the explosive material is peroxide-containing or nitrate-containing.

34. The method of claim 30, wherein the explosive material is TATP, HMTD, or PETN.

35. The method of claim 30, wherein the organic compound is benzene, pthalates, mycotoxins, tetrahydrofuran, acetone, alkanes, alcohols or toluene.

36. A system for detecting an explosive material comprising the sensor of claim 1.

37. A system for detecting a material that can turn into gaseous form comprising the sensor of claim 1.

38. A system for detecting a material that is volatile or semi-volatile comprising the sensor of claim 1.
